(19) United States
(12) Patent Application Publication
(10) Pub. No.: US 2010/0055157 A1
(43) Pub. Date: Mar. 4, 2010

(54) SILVER BASED ANTIMICROBIAL COMPOSITIONS AND ARTICLES

(75) Inventor: Valerie E. GUNN, Cape Neddick, ME (US)

Correspondence Address: WILMERHALE/BOSTON, 60 STATE STREET, BOSTON, MA, 02109, US

(73) Assignee: ANDOVER HEALTHCARE, INC., Salisbury, MA (US)

(21) Appl. No.: 12/549,749

(22) Filed: Aug. 28, 2009

Related U.S. Application Data
(60) Provisional application No. 61/190,432, filed on Aug. 28, 2008.

(51) Int. Cl. A61K 33/38; A61L 15/16; A61P 31/00

(52) U.S. Cl. 424/447; 424/618

(57) ABSTRACT

Antimicrobial articles, including cohesive, adhesive, and pressure-sensitive adhesive articles, comprising a substrate and a silver compound, and methods of making antimicrobial articles are described.

### CROSS REFERENCE TO RELATED APPLICATIONS

This application claims the benefit of U.S. Provisional Application No. 61/190,432, filed Aug. 28, 2008, the entire contents of which are hereby incorporated by reference herein.

### BACKGROUND OF THE INVENTION

Humans and other animals are in a constant immune-system battle with agents of infectious disease, including bacteria and viruses, as well as pathogenic fungi and protozoa. These agents of infectious disease reside in the environment, and in the flora of the skin. A particular problem for healthcare professionals dealing with these infectious agents has been the development of antibiotic resistant bacteria, which are refractory to many of the antibiotic agents that initially promised to provide a reliable cure. Indeed, the Center for Disease Control (CDC) has recently made the issues of combating antimicrobial resistance and preventing emerging infectious diseases two of its top priorities (see “Federal Register Notice on Draft Public Health Action Plan to Combat Antimicrobial Resistance” (2000) JAMA 284:434; (2000) MMWR 49:603; and “Preventing Emerging Infectious Diseases” published by the National Center for Infectious Diseases, Centers for Disease Control and Prevention, Atlanta, Ga.).

A particular problem for the healthcare industry has been the development and spread of infections, specifically those caused by Staphylococcus aureus (including MRSa) within the hospital environment. Medical devices, such as intravascular catheters provide a method for delivering fluids, medications, and nutrients to patients; however, their use is also frequently associated with Hospital Acquired Infections (HAIs). Adhesive tapes used in conjunction with catheters and other medical devices are uniquely vulnerable to facilitating the spread of such infections in hospitals. This is because they are generally not washed or sterilized once they have been unpackaged, and, further, because a single roll of tape is generally used by several clinicians and on many different patients, and thereby becomes exposed to many different individuals. Further, such adhesive tapes are frequently handled using ungloved hands and applied in close contact to the intravascular insertion site for extended periods of time. Indeed, one study found surprisingly high levels of infectious bacteria, including all forms of Staphylococcus aureus, on the outer layer and the sides of rolls of medical tape (3M Transpore™) that were in use throughout a hospital in Toronto (see Redelmeier and Livesley (1999) J. Gen. Int. Med. 14: 373-5).

### SUMMARY OF THE INVENTION

The invention is based, at least in part, on the surprising discovery that a glass bead containing silver can be added to a cohesive article, resulting in an article that retains cohesive as well as antimicrobial properties. Accordingly, in one aspect, the invention features an antimicrobial article comprising a substrate and a silver compound, wherein the silver compound is present in an amount sufficient to treat an infectious agent through contact of the antimicrobial article with a subject. In some embodiments, the silver compound is a glass bead containing silver described herein, and the substrate comprises a formulation comprising the silver compound. In some embodiments, the formulation comprises a cohesive agent, an adhesive agent, or a pressure-sensitive adhesive agent described herein. In some embodiments, the article is a tape. In other embodiments, the article is a bandage.

In another aspect, the invention features a method of making an antimicrobial article, the method comprising adding a silver compound-containing resin to an article in an amount sufficient to treat an infectious agent through contact of the antimicrobial article with a subject. In some embodiments, the silver compound is a glass bead containing silver described herein. In some embodiments, the resin comprises a cohesive agent, an adhesive agent, or a pressure-sensitive adhesive agent described herein. In some embodiments, the article is a tape. In other embodiments, the article is a bandage.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein, are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

### DETAILED DESCRIPTION OF THE INVENTION

The invention is based, at least in part, on the discovery that a glass bead containing silver can be added to a cohesive article. Surprisingly, it was found that such an article retained cohesive properties, and that the glass bead containing silver incorporated into the article retained antimicrobial properties. Accordingly, the invention provides cohesive and adhesive, including pressure-sensitive adhesive, formulations into which the antimicrobial compositions described herein are incorporated.

### Antimicrobial Compositions

The compositions and methods described herein include at least one antimicrobial composition, e.g., a silver composition. The term “silver composition” encompasses compounds such as ion-exchange resins, zeolites, substituted glass compounds, and the like, that release silver metal ion bonded thereto upon the presence of an anionic species. One exemplary silver composition is IonPure® (Ishizuka Glass, Iwakura-shi, Japan), such as IonPure WPA (≦10 microns), IonPure WPA (≦40 microns), IonPure IZA (≦10 microns), and IonPure IPM (≦50 microns). Particular embodiments include the use of glass-containing silver zeolite compositions capable of releasing the silver ions. Another exemplary silver composition is ACT Z 200′ and ACT T 558′ (EnviroCare Inc., Wilmington, Mass., USA). Particular embodiments include the use of these zeolite compositions capable of releasing the silver ions.

Other silver compositions include AlphaSan® (Milliken & Company, Spartanburg, S.C.); Agion® natural zeolites (Agion Technologies, Inc., Wakefield, Mass.); Zeomic® AJ (Sinanen Zeomic Co., Tokyo, Japan); Apacider® (Sangi Co., Tokyo, Japan); silver metal coated nano-spheres, fibers, or particles; and polymeric ligands. Various combinations of these silver compositions can be used to control the silver release rate over time.

### Antimicrobial Articles

An antimicrobial composition described herein can be incorporated into cohesive and adhesive, including pressure-sensitive adhesive, formulations to produce antimicrobial articles. Such antimicrobial articles include, for example, articles for topical/cutaneous contact with a subject (e.g., tapes and bandages). Exemplary articles include tapes and bandages and may be constructed of any number of materials woven and non-woven fabrics, knit fabrics and films, including porous films (exemplary porous films are described in U.S. Ser. No. 11/204,736). The antimicrobial articles described herein may be used in any suitable application, e.g., in sports or medicine. Exemplary articles for topical/cutaneous contact that can be used in the compositions and methods described herein are known in the art and described below and in, e.g., U.S. Pat. No. 5,762,623 and U.S. Publ. Nos. 20040214494; 20050158539; and 20050249791, the contents of which are incorporated herein by reference in their entirety.

Any of the cohesive, adhesive, or pressure-sensitive adhesive articles described herein can include an antimicrobial composition described herein. In some embodiments, the antimicrobial composition is mixed with a cohesive, elastic, or pressure-sensitive adhesive formulation prior to the forming of the article. In other embodiments, the antimicrobial composition can be mixed with a resinous material to produce an antimicrobial resin, which can be coated onto one or more surfaces of an article described herein.

In particular embodiments, the articles are Cotton Arun 150; 151; 170; poly-cotton Arun 112; polyester; polyamide; Cerex; warp-knits (Milliken, Jinda) and non-wovens such as CL, KT, RG, High Tech, and FQN. In some embodiments, the article is a cohesive elastic bandage such as CoFlex, CoFlex NL, CoFlex LF, Coflex LF2, PowerFlex, PetFlex, CoFlex Medical Tape, Trainer's Tape, Moleskin, PowerTape, ROM Tape, Surgical Tape, and PowerFast. In other embodiments, the article is a cohesive Tape such as PowerTape. In other embodiments, the article is a pressure sensitive tape or bandage such as Medical Tape, Trainer's Tape, Moleskin, PowerFast, and Surgical Tape. In yet other embodiments, the article is an island dressing such as Andover's Absorbant Foam Dressing and any other known dressing.

The antimicrobial resin can be chemically similar or dissimilar to the functional chemistry at the surface of the article. For example, the antimicrobial resin can be formed from a cohesive resin that is the same cohesive composition used to form a cohesive article described herein. In other situations, the antimicrobial resin can be formed from a cohesive resin that is different from the cohesive composition used to form a cohesive article described herein.

In some embodiments, a Polychloroprene (such as Neoprene 654, Neoprene750, Dispercoll C74, Dispercoll C-84) or a Vinyl acetate ethylene (such as Airflex 323, Airflex 400, Airflex 410, Airflex 405, Airflex 421, Airflex 920) can be used to form an antimicrobial resin. Other antimicrobial resins can be made from natural rubber latex, butadiene, isoprene, Acrylonitrile, and combinations with styrene, polyurethane, any PSA, acrylic, carboxylated styrene butadiene rubber, silicone, fluorocarbon, microcrystalline waxes, and Interpenetrating Polymer Networks (IPN's) of Si-PUR.

### Methods of Making Antimicrobial Articles

Any number of methods can be used to contact a coating of an antimicrobial resin describe herein to one or more surfaces of a flexible substrate. The coating thickness can be determined by the concentration (wt/wt) of the film forming resin in the total solution. Such coating methods include, but are not limited to, spray, “dip and nip”, knife over roll, reverse Meyer rod, reverse gravure, kiss coating, printing, or by the Chemical Foam System developed by Gaston Systems Inc. (EP 0 995 826 B1).

Some methods include the use of dispersing agents, wetting agents, or rheology modifiers. Non-limiting examples of dispersants or wetting agents include Zetasperse 1200, Zetasperse 1400, Zetasperse 1600, Zetasperse 2300, for example at 0.5% or 1.0%. Non-limiting examples of rheology modifiers include ASE 60, Rheolate 360, and Paragum 184. The target coating weight of the antimicrobial resin can be greater than about 0.1 gsm to about 10 gsm.

### Cohesive Articles

The antimicrobial compositions described herein can be used on a variety of substrates. For example, a cohesive composition can be used to form cohesive articles, e.g., those described in U.S. Pat. Nos. 6,156,424 and 5,762,623, the contents of which are incorporated by reference herein. For example, a cohesive composition can be used on a substrate of the type sold by Andover Healthcare, Inc. (formerly known as Andover Coated Products Inc.) of Salisbury, Mass. under the trademark “POWERFLEX” and described in U.S. Pat. No. 5,762,623. As described in this patent, the substrate includes a plurality of longitudinally-extending elastic threads or yarns sandwiched between a layer of a warp-knit weft-insertion fabric and a layer of a non-woven fabric.

The substrates can be made of any of a wide range of materials, and may have a wide range of structures. For example, the substrate can have one or more layers, each of which can be, e.g., a woven, knitted, warp-knit weft-insertion or non-woven fabric, or paper. The substrate can also be a surface-treated polymeric, such as a sheet of linear, low-density polyethylene (“LLDPE”) or linear, low-density polypropylene (“LLDPP”), one or more surface of which has been treated to insure adhesion to the cohesive composition. Similarly, the substrate structure can be elasticized, either by knitting or weaving elastic threads into one or more of the layers, or by knitting or sewing elastomeric threads through a single or multi-layer substrate.

In instances in which the cohesive article is a tape or bandage, the substrate can include a woven, knitted, or warp-knit weft insertion fabric, or a non-woven fabric such as a non-woven scrim, of either natural or synthetic fiber. In one example, the substrate includes a single layer of a non-woven fabric through which threads are knitted and a cohesive composition described herein is deposited on opposite sides of the fabric by, e.g., spraying or coating. In some situations, the substrate is a tape or bandage that includes nylon or polyester or polypropylene.

In other examples, the substrate of the tape or bandage includes a first layer and a second layer of non-woven fabric and a third layer that is elastic in a direction extending longitudinally of the tape or bandage, where the third layer is positioned between, or knitted or woven within, the first layer and the second layer of non-woven fabric.

In other examples, the substrate of the tape or bandage includes the following: a first layer of warp-knitted weft insertion fabric oriented with the knit yarns extending longitudinally of the tape or bandage; a second layer of a non-woven fabric; and a third layer that is elastic in a direction extending longitudinally of the tape or bandage, where the third layer is positioned between, or knitted or woven within, the first layer and the second layer.

It is well known to make tapes or bandages in which the cohesive composition is natural rubber latex. How to make such tapes or bandages is described in the prior art, e.g., U.S. Pat. No. 5,718,674, the substance of which is hereby incorporated by reference. In general, such tapes or bandages are made from a water-based emulsion of a natural rubber latex to which a tackifier has been added. The resulting latex/tackifier structure is applied to the substrate (typically by saturating the substrate with the emulsion or coating the emulsion onto the opposite sides of the substrate), and the structure is then dried to produce the desired end product.

### Methods of Determining Cohesive Bond Strength

The cohesive bond strength of a cohesive article described herein can be determined by known methods, such as a T-Peel test and a shear bond test. A T-Peel Test can be performed using, e.g., two strips of a finished cohesive article measuring 1 inch in width and of equal length. The two strips are placed face to face and a cylindrical weight is rolled across the surface of the superimposed strips. The two non-superimposed ends are clamped in the jaws of a tensile testing apparatus and pulled linearly in opposite directions pulling the two strips apart. The resistance of the superimposed strips to the movement of the clamps is typically measured in oz/inch of width. In certain situations, the cohesive articles described herein have a T-Peel ≧10 oz/in.

A shear bond test can be performed using, e.g., two strips of a finished cohesive article measuring 1 inch in width and 5 inches in length. The two strips are placed linearly so the end of one strip overlaps the end of another strip by 2 inches lengthwise. A cylindrical weight is rolled across the surface of the superimposed end of the two strips. The non-superimposed end of the two strips are clamped in the jaws of a tensile testing apparatus and pulled linearly in opposite directions. The strength of the shear bond of the superimposed ends is typically measured in oz/in². In some instances, the cohesive articles described herein have a shear modulus ≧10 oz/2 in².

### Foam Layer Cohesive Articles

The cohesive compositions described herein can be used in foam layer cohesive articles, for example medical bandages and wraps. In some instances, the foam layer cohesive articles include a foam layer, and optionally include one or more additional layers, such as an elastic layer, or a fabric, which can provide enhanced elasticity, strength, softness and/or cohesion. The articles typically have first and second oppositely-facing exterior surfaces, and in some instances both of these first and second surfaces are at least partially coated with a cohesive composition described herein. In various situations, the cohesive composition substantially permeates the foam and secures the foam layer to other layers within the article. However, in other situations a cohesive composition described herein does not permeate the foam or other layers, but coats at least a portion of one or both of the major exterior surfaces of the article. The article can also include a foam pad that can be applied to a wound. The article can be wound upon itself to form front to back oriented layers.

The presence of the foam layer is useful in many respects. For example, if the article is used as a wrap, the foam layer can provide enhanced comfort and softness relative to bandages that do not include a foam layer. In addition, in situations where the foam layer defines at least a portion of one of the major exterior surfaces of the article, the microscopic structure of the foam can enhance the cohesive properties of the article. For example, the foam layer can include a plurality of open cells that have surfaces facing the exterior of the article, and the cohesive composition can coat these open cell surfaces without filling the cells. The open cells can appear to form tiny, outward-facing “suction cups.” If these suction cups are compressed against a surface, e.g., against another surface of the article if the article is wound around a body part, or against a non-porous surface of a medical device being affixed to a body part, the “suction cups” may form a partial vacuum that imparts a particularly secure cohesive property to the article. It has been observed that if the article gets wet while it is wrapped around a body part, it does not unravel as conventional latex free cohesive bandages could, but rather maintains the secure fit around the body part.

In some instances, the foam layer need not define the entirety of one of the major exterior surfaces of the article in order to provide the article with enhanced cohesion. For example, a porous fabric (such as a woven scrim, among others) can be applied over the foam layer. This fabric can be sufficiently porous such that the foam layer is exposed through the fabric. Without wishing to be bound by theory, this allows at least some of the exposed open cells on the surface of the foam layer, in conjunction with the fabric coated layer, to behave as tiny “suction cups” when the fabric-coated foam layer is compressed against a surface, and thus maintaining at least some of the enhanced cohesion of the article. While in some situations, the presence of the porous fabric may reduce the enhancement in cohesion compared to a fabric-free embodiment, the porous fabric can impart other useful properties (e.g., enhancing the strength of the article, allowing the article to be more uniformly torn by hand, and/or providing a desired hand-feel to the article).

The foam layer can also include at least some closed cells, or even have a substantially entirely closed-cell structure. The closed cells will not necessarily provide a comparable “suction cup” action to the open cells, but the foam will still impart a soft feel to the article.

In one illustrative example of a foam layer cohesive article, the article includes a backing layer of warp-knitted weft-insertion fabric, a bottom layer of polyurethane foam, and a middle layer of longitudinally-extending, transversely spaced (e.g., about 12 per inch) elastic strands. The three-layer structure can be laminated together with a cohesive composition described herein that impregnates all three layers. The cohesive composition substantially coats the major exterior surfaces of the article, and also permeates all three layers, thus securing them to each other.

In use, the foam layer is inherently elastic, i.e., it can be deformed extensively and then substantially return to its original shape. Thus, in some instances, the presence of the elastic strands is not necessary. However, in certain applications, the presence of the elastic may enhance the compression the article can exert if, e.g., the article is wound around a body part, may add strength to the wrap, and may lead to more rapid recovery of the article to its original shape after stretching.

In other instances, the backing layer is not a warp-knit weft-insertion fabric. Rather, in general, a variety of different layers (or no layer at all) can be used in the backing. The backing layer may include a plurality of layers. For example, the backing can include an elastic fabric, which may include elastic yarns woven throughout the fabric. In this case a separate elastic layer may not be necessary, but it can still be included if desired depending on the application. The backing layer can also include a non-woven fabric. For example, situations in which an open-cell foam layer defines a first major exterior surface of the article, and in which a non-woven fabric is used as a backing and thus defines a second major exterior surface of the article, have been found to be particularly cohesive when the foam layer is compressed against the non-woven fabric backing. Without wishing to be bound by theory, it is believed that the non-woven fabric backing can provide an enhanced surface area relative to some other kinds of fabrics, and/or may be sufficiently open that some of the open-cell “suction cups” of the foam layer underlying the non-woven fabric are available for use. Knit fabrics, e.g., chain knits, circular knits, or warp-knit weft-insertion fabrics, can also be used in the backing layer. Woven fabrics, e.g., woven scrims or open mesh fabrics, can be used. In some embodiments, one or more of the layers used in the backing is substantially porous, e.g., has about a 25% to 75% open structure, e.g., about 50% open.

The layer(s) used in the backing layer are not limited to fabric-based layers. For example, in some instances, a second foam layer is used in the backing. The second foam layer can provide enhanced comfort, as well as a stronger peel strength. This can result in an enhanced grip, for example if the article is used on a hand.

As mentioned above, not all articles include backing and/or elastic layers, as the foam layer itself provides many useful properties, such as cohesion, softness, and strength, when coated with a cohesive composition described herein in the absence of other layers.

Also, as mentioned above, an additional layer, e.g., a fabric layer, can be added to the front of the foam, e.g., in addition to a backing layer added to the back of the foam. The front additional layer can include a fabric described herein, and/or can include an elastic layer. The backing and/or front fabrics can also have different strengths in the machine and cross directions to provide facile and even hand-tear to the foam layer cohesive article.

In some instances, the backing can be considered to be both the fabric layer and the elastic layer together. The term “backing” or “second layer” should not be construed as being limited to a single-ply layer, but in fact can be multiple-ply and have many layers. The backing can be secured to the foam layer using a cohesive composition described herein, for example, by permeating the foam and the backing with a cohesive composition that binds the layers together when it dries.

Some combinations of layers that can be used to form various types of foam layer cohesive articles are listed below. The first listed layer defines at least a portion of the first major exterior surface of the article, the last listed layer defines at least a portion of the second major exterior surface of the article, and any layers in between are presented in the order listed and may themselves define at least a portion of the first and/or second major surfaces of the article, depending on the porosity of any intervening layers. The listed types are not intended to be limiting, or inclusive of all possible examples:
Warp-knit weft-insertion fabric layer, elastic layer, foam layer.
Warp-knit weft-insertion fabric layer that is pre-coated with color, elastic layer, foam layer.
Warp-knit weft-insertion fabric layer, foam layer.
Warp-knit weft-insertion fabric layer, foam layer, warp-knit weft-insertion fabric layer.
Warp-knit weft-insertion fabric layer, elastic layer, foam layer, warp-knit weft-insertion fabric layer.
Warp-knit weft-insertion fabric layer, elastic layer, non-woven fabric layer, foam layer.
Foam layer, warp-knit weft-insertion fabric layer, elastic layer, foam layer.
Foam layer, elastic layer, non-woven fabric layer.
Foam layer, elastic layer, foam layer.
Foam layer, elastic layer.
Foam layer, warp-knit weft-insertion fabric layer, foam layer.
Foam layer, elastic layer, woven fabric layer, foam layer.
Foam.
Non-woven fabric layer, elastic layer, foam layer.
Non-woven fabric layer, woven fabric layer, elastic layer, foam layer.
Non-woven fabric layer, elastic layer, non-woven fabric layer.
Open mesh fabric layer, foam.

The article can be wound into a roll. In some situations, the first major exterior surface of the article is wound onto and cohesively attaches to the second major exterior surface of the article, or vice versa. In other instances, a removable release layer is placed in between the major exterior surfaces of the article. Preferably, the release layer is not cohesive, but readily detaches from the major exterior surfaces of the article. A release layer can be useful, e.g., in circumstances where cohesion between the major exterior surfaces of the article is relatively high, and the presence of the release layer would facilitate unwinding of the rolled article or otherwise facilitate use of the article. In some situations, a release layer is with an article that is not rolled.

Specific details of different kinds of useful fabrics, elastic layers, cohesive compositions, foam layers, and the like can be found below. Additionally, those of ordinary skill in the art will recognize that other layers and compositions can be used.

Characteristics of Exemplary Embodiments of Foam Layer Cohesive Articles

In many situations, the foam layer cohesive articles provide secure cohesive bonds, e.g., when the front foam layer is bonded to the backing layer back of the article, e.g., when the article is wound upon itself to form front to back oriented layers, either on the roll or if it is used to wrap a body part. In some articles, the strength of this secure cohesive bond between front to back oriented layers of the article is characterized by a peel force bond strength of, e.g., between about 5 oz/in-w and about 40 oz/in-w as measured in a standard peel force test, depending on the particular application and configuration, e.g., ratio of open cells to closed cells in the foam, the presence of additional layers, and the cohesive composition. In some instances, the peel bond force strength can be between about 12 oz/in-w and about 35 oz/in-w, between about 20 oz/in-w and about 30 oz/in-w, or about 25 oz/in-w in a standard peel force test. That such peel force bond strengths can be achieved in latex free articles is particularly surprising.

In some articles, the secure cohesive bond provided by the foam layer front of the article is characterized by a shear force bond strength of about 2 lb/in² to about 30 lb/in² in a standard shear force strength test to a stand surface substrate, depending on the particular application and configuration as mentioned above. In some situations, the article can have a shear force bond strength of between about 5 lb/in² and about 20 lb/in², or between about 9 lb/in² and about 15 lb/in², or between about 11 lb/in² and about 13 lb/in², or about 12 lb/in² in a standard shear force strength test.

In some articles, the overall laminated elastic article is characterized by the ability to stretch about 50% to about 200% beyond its original unstretched length before it fails. The inherent elasticity of the foam and of other layers that can be present determine, in part, the article's ability to stretch before failure. For example, the presence of a fabric (e.g., a warp-knit weft-insertion fabric) can prevent the article from stretching as far as it otherwise would be able to, because the yarns of the fabric may themselves not be extensible. Thus, the weave of the fabric may limit the extensibility of the article. As discussed below, the fabric may be “gathered” during fabrication so that the article is extensible to a desired percent stretch before reaching the maximum extension of the fabric, at which point further stretch would at least partially damage the article. In particular instances, the article has a percent stretch of about 100% to about 180%, or about 120% to about 160%, or about 140%, beyond the unstretched length before failure.

In some situations, the overall article is characterized by having a tensile strength of about 8 lb/inch to about 25 lb/inch, e.g., about 12 lb/inch, in a standard tensile strength test. In other instances, the article is characterized by having an overall weight of about 30 g/m² to about 100 g/m², with the cohesive composition making up about 20% to about 70% of this overall weight. In certain instances, the article has an overall weight of about 40 g/m² to about 80 g/m², with the cohesive composition making up about 25% to about 45% of this overall weight. In one example, the article has an overall weight of about 60 g/m², and the cohesive composition makes up about 35% of the overall weight.

Apparatus and Methods of Making Foam Layer Cohesive Articles

An exemplary apparatus for preparing a foam layer cohesive article is described in co-pending U.S. application Ser. Nos. 11/809,738; 11/809,766; and 11/809,469 (all of which are incorporated by reference). This exemplary apparatus includes three separate feed rolls for supplying a foam layer, warp-knit weft-insertion fabric backing layer, and an elastic layer, e.g., elastic yarns. The elastic layer is fed between the foam layer and the warp-knit weft-insertion fabric backing layer. The foam layer, the warp-knit weft-insertion fabric backing layer, and the elastic layer are guided together into nip rolls that supply a metered amount of a cohesive composition, e.g., a cohesive composition described herein, to the layers from a reservoir. In many instances, the cohesive composition is of a solids content and viscosity that permits impregnation and coating of the foam base and warp-knit weft-insertion fabric backing layers of the article. Additives, e.g., antifoaming agents, can be added to improve the processability of the cohesive formulation.

In certain situations, the backing layer is fully extended and the foam and the elastic layer are stretched when they are laminated together with the cohesive composition. For example, the elastic layer can be stretched by about 50% to about 250%, or about 130% to about 170%, or about 150% of its original unstretched length when it is laminated to the backing layer and the foam layer. The foam layer can be stretched by about 0% to about 20% when it is laminated to the elastic layer and the backing layer, or can be fully extended (but not stretched) when it is laminated to the elastic layer and the permeated backing layer. After passing through the nip rolls, which supply compression to the layered article, the layers can be further laminated together by passing between an infrared heater and a heated plate maintained at an appropriate temperature. The heater can be, e.g., heated air, heat lamps, or any other conventional source of heat. The laminate structure then is passed through multiple rollers to dry the laminated structure and to secure the warp knit fabric backing to foam layer front of the article. In many instances, essentially all of the carrier liquid is removed in the drying step, and the finished product is then wound into a take-up roll. The take-up roll can then be used directly or rewound into a finished roll of any desired length, width and winding tension.

Note that different embodiments of the foam layer cohesive articles can be fabricated using modifications of the apparatus described herein, or with entirely different machinery and/or methods. For example, if the article does not include a backing layer and/or elastic layer, those reels and steps can be omitted. Or, for example, the backing layer and/or the foam layer can be pre-coated with the cohesive composition, and the elastic layer positioned between the pre-coated woven backing layer and the pre-coated foam layer. The backing layer pre-coated with the cohesive composition may include a fabric, e.g., a woven material, permeated with a binder (such as acrylic nitrile) and then coated with a cohesive composition described herein.

In one illustrative example, a foam layer cohesive article for use, e.g., as a tape or a bandage, can be fabricated by permeating a backing layer of warp-knit, weft-insertion polyester fabric with a cohesive composition described herein. The cohesive composition is also used to permeate a foam layer of open cell polyurethane foam material having a density of about 1.40 lb/ft³, a thickness of about 0.025 inches, and weight of about 22 g/m². The cohesive-permeated backing layer is then laminated to the cohesive-permeated foam layer along with an elastic layer that is positioned between the permeated backing layer and the permeated foam layer. The elastic layer laminated between the permeated backing layer and the permeated foam layer is made up of elastic spandex yarns having a denier of about 210, a percent stretch of about 700% to about 800% beyond their unstretched length before failure, and a weight of about 6.5 g/m² of the overall article. Finally, the resulting laminated article is dried to produce a foam layer cohesive article that can be formed into a roll or used directly.

Foam Layer

In some articles, the foam layer is a cellular sheet material formed of a suitable material, e.g., chemically foamed or aerated plastic material, foamed rubber or a non-hardening cellulose sponge material. In some articles, the foam layer includes a plurality of open cells that behave as tiny “suction cups” that enhance the cohesiveness of the article. These open cells can define at least a portion of one of the major exterior surfaces of the article. In some articles, the foam layer includes a plurality of closed cells. The closed cells do not necessarily provide as strong a “suction cup” effect as open cells would; however, the closed cells provide enhanced cohesion and comfort relative to a foam-free product. The cohesion of the article, as well as the adhesion of the article to other surfaces (such as the non-porous surfaces of braces or other medical equipment) can be adjusted by, among other things, selecting the ratio of open cells to closed cells in the article, as well as adjusting the cohesive composition appropriately.

Open cell foams and closed cell foams are well known in the art, and those of ordinary skill in the art will recognize that foams termed “open cell” will naturally include some closed cells, and that foams termed “closed cell” will naturally include some open cells. Thus the terms “open cell” and “closed cell” do not imply that the foam must necessarily include 100% open or 100% closed cells. In general, in closed cell foams most of the cells are closed off from each other, and water absorption is low. Open-cell foams have an interconnecting cell structure, absorb liquids, are generally softer than closed-cell foams, and have less structural integrity than open cell foams.

In some situations, the foam material includes one or more of polyurethane, polyester, polyester/polyurethane and polyethylene. When incorporated into the article, the layer can have a weight of about 18 g/m² to about 30 g/m² of the article. In particular, the foam layer can have a weight of about 22 g/m² of the article. When constructed of polyurethane, the foam layer can have a density of about 1.00 lb/ft³ to about 3.00 lb/ft³, e.g., about 1.40 lb/ft³. The foam layer can have a thickness of about 0.01 inch to about 0.25 inch, e.g., about 0.025 inch to about 0.035 inch. The foam layer can be of any thickness desired for a particular application. In general, the greater the thickness, the greater the cushioning effect; however, a greater thickness also increases the bulk of the article so the appropriate thickness will depend on the particular use. For example, a thinner foam may be useful for arm or leg wounds in which clothes would be worn over the wrapped article. On the other hand, a thicker foam may be useful where applied over a bruise (since it would provide more cushioning) or for use with animals (in which case the wrapped article would be likely to experience additional wear).

In some articles, the foam layer is a thin-gauge sheet of polyurethane or polyester/polyurethane foam material having a thickness on the order of 0.025 inches. One suitable polyester-polyurethane foam sheeting material is product number S82F polyester polyurethane foam (W.T. Burnett & Co., Jessup, Md.). This foam sheeting material has a density of about 1.4±10% lb/ft³, a minimum tensile strength of 22.0 psi, an average tensile strength of 30.0 psi, a minimum tear resistance of 3.00 pli, an average tear resistance of 4.00 pli, and a minimum elongation of 300% (average of 400%) (as determined by the ASTM-D3574 standard methods of testing flexible cellular materials—slab, bonded and molded urethane foam). The S82F polyester polyurethane foam has a minimum compression force deflection of 0.35 psi and an average compression force of 0.50 psi at 25% deflection; a minimum compression force deflection of 0.40 psi at 50% deflection, and an average compression force of 0.55 psi at 25% deflection. Although the S82F polyester polyurethane foam having a thickness of 0.025 inches produces a laminated article with satisfactory cohesive and cushioning properties, other thicknesses (e.g., up to 0.10 inch or even greater) can be employed to provide additional cushioning.

Other exemplary materials suitable for use as a foam layer include a flexible foamed polyester material, which may provide enhanced flame resistance. Alternatively, foamed rubber sheeting or non-hardening cellulose sponge sheeting can be used as the core, either in combination with or in substitution for sheeting of foamed plastics material. Other alternatives for the foam layer include a sheet of a suitable foamed thermosetting material, or foamed rubber sheeting, or, non-hardening cellulose sponge sheeting. Additionally, the foamed material can incorporate fire retardant or suppressant agents, which may be selected to resist leaching during normal wear or exposure to the elements to which the article is likely to be subjected.

In some articles, the foam layer is fabricated or commercially purchased with a plurality of open cells on at least one of its major surfaces. At least some of the open cells remain open during fabrication of the article, even after permeation with the cohesive composition and lamination to other layer(s). The open cells then act as “suction cups” and thus enhance the cohesiveness of the article. In other articles, the foam layer is fabricated or commercially purchased with a plurality of closed cells. During the lamination operation a number of the closed cells may be partially severed and opened. In some articles, the foam layer as fabricated or purchased has a cell size of the individual cells that is maintained below a determined maximum, and a preponderance of the cells are of smaller size and extent than the size of the largest of the cells.

Front and/or Backing Layers

As described above, the front and/or backing layers of the article can include, without limitation, a second foam layer, an elastic layer, an elastic fabric, a knit fabric, a woven fabric, or a nonwoven fabric. Although this section generally describes backing layers, the description applies equally to front layers, applied to the other side of the foam layer.

In some articles, a backing layer described herein can include one or more layers that facilitate hand-tearing of the article, and/or provide the article with suitable longitudinal tensile strength for use in applications such as, e.g., wrapping a limb or other body part, or any other suitable application.

In some articles, the backing layer can be a warp-knit weft-insertion fabric. In particular warp-knit weft-insertion fabrics, the warp yarns can include a plurality of longitudinally-spaced knitted loops through which the weft yarns extend transversely of the article. The warp yarn(s) can be of lower tensile strength than the weft yarn(s) so as to facilitate hand tear, but the relative strengths of the overall article in the machine direction versus the cross direction can also be influenced by the density of the warp and weft yarns. Accordingly, the overall strength of the article in the machine direction may be higher than that in the cross direction, despite the use of a weft yarn having a higher denier than that of the warp yarn.

The warp yarns and weft yarns of the warp-knit weft-insertion fabric can be yarns of any suitable material. For example, the warp yarns and weft yarns can be yarns of polyolefin, polyester, poly-cotton, cotton, or any other suitable material that allows for hand-tearing of the article and provides the desired tensile strength. The weft yarns extending transversely of the article can be, for example, textured filament yarns.

The warp yarns of the warp-knit weft-insertion backing layer can be spaced at a density in the range of about 9 yarns per inch to about 48 yarns per inch, as measured transversely of the article. In some articles, the warp yarns can be spaced at a density in the range of about 12 yarns per inch to about 24 yarns per inch, particularly at a density of about 18 yarns per inch. In other articles, the warp yarns can be spaced at a density in the range of about 18 yarns per inch to about 30 yarns per inch, about 30 yarns per inch to about 48 yarns per inch, or any other suitable range of densities. The warp yarns of a warp-knit weft-insertion backing layer can have a denier in the range of about 20 to about 80. In some articles, the warp yarns can have a denier in the range of about 30. In other articles, the warp yarns can have a denier in the range of about 20 to about 60, about 40 to about 80, about 60 to about 100, or any other suitable range of deniers.

The weft yarns of the warp-knit weft-insertion backing layer can be spaced at a density in the range of about 6 yarns per inch to about 48 yarns per inch, as measured longitudinally of the article. In some articles, the weft yarns can be spaced at a density in the range of about 9 yarns per inch to about 18 yarns per inch as measured longitudinally of the article, particularly at a density of about 12 yarns per inch. In other articles, the weft yarns can be spaced at a density in the range of about 6 yarns per inch to about 24 yarns per inch, about 18 yarns per inch to about 36 yarns per inch, about 30 yarns per inch to about 48 yarns per inch, or any other suitable range of densities. The weft yarns of the warp-knit weft-insertion backing layer can have a denier in the range of about 50 to about 200. In some articles, the weft yarns can have a denier in the range of about 60 to about 100, particularly a denier of about 70. In other articles, the weft yarns can have a denier in the range of about 40 to about 170, about 170 to about 300, or any other suitable range of deniers.

In some articles, the warp-knit weft-insertion backing layer can have a weight of not more than about 50 g/m². In some articles, the warp-knit weft-insertion backing layer can have a weight in the range of about 10 g/m² to about 20 g/m², particularly about 15 g/m². In other articles, the warp-knit weft-insertion backing layer can have a weight in the range of about 10 g/m² to about 30 g/m², about 20 g/m² to about 50 g/m², or any other suitable range of weights.

An illustrative fabric that can be used for the warp-knit weft-insertion fabric is style number 071355 obtained from Milliken & Company of Spartanburg, S.C. (“the 18×12 Milliken fabric”). This Milliken fabric is a polyester warp-knitted weft-insertion fabric having a warp denier of about 30 and a weft denier of about 70. This Milliken fabric weighs approximately 0.33 ounces per square yard, has warp yarns spaced at about 18 yarns per inch, weft yarns spaced at about 12 yarns per inch, and a tensile strength of about 11 lb/inch (machine direction).

Another illustrative fabric that can be used for the warp-knit weft-insertion fabric is style number 997590 (pattern # 550) obtained from Milliken & Company of Spartanburg, S.C. (“the 18×18 Milliken fabric”). This Milliken fabric is a polyester warp-knitted weft-insertion fabric having a warp denier of about 30 and a weft denier of about 70. This Milliken fabric weighs approximately 14.4 g/m², has warp yarns spaced at about 18 yarns per inch, weft yarns spaced at about 18 yarns per inch, and a tensile strength of about 11 lb/inch (machine direction).

Another exemplary warp-knit weft-insertion fabric is style number J477 obtained from Chima, Inc. of Reading, Pa. (“Chima fabric”). The Chima fabric is a polyester warp-knitted weft-insertion fabric having a warp denier of about 50 and a weft denier of about 150. The Chima fabric weighs approximately 0.74 ounces per square yard, and has a tensile strength of about 22 lb/inch.

The backing layer of the article can also include a woven scrim fabric. A “scrim” fabric is a loose plain-woven fabric, frequently of cotton, with fine to coarse mesh. Scrim woven fabrics also have warp (machine direction) yarns and weft (cross direction) yarns, with adjacent warp yarns extending longitudinally on opposing sides of the plane defined by weft yarns in a non-looped fashion. An illustrative scrim fabric is style number 013228400011 obtained from DeRoyal Textiles of Camden, S.C. (“DeRoyal fabric”). The DeRoyal fabric is a cotton scrim woven fabric having a warp yarn density of about 32 yarns per inch measured transversely of the article and a weft yarn density of about 28 yarns per inch measured longitudinally of the article. The DeRoyal fabric weighs approximately 1.31 ounces per square yard. Still other examples of fabrics that can be used for the warp-knit weft-insertion backing layer include greige cloth and other such scrim woven fabrics known in the art.

The backing layer can include a nonwoven layer of material. The fibers of a nonwoven material are intimately entangled with each other to form a coherent, breathable fibrous material. Nonwoven materials that can be used include, e.g., a synthetic spunbonded nonwoven material, a spun-melted nonwoven material, a wet laid nonwoven material, a dry laid nonwoven material, a needle punched nonwoven material, or a melt blown nonwoven material. Nonwoven material can be constructed using any suitable fiber composition, e.g., nylon, polyester, polypropylene, rayon, cellulosic, polyamide, acrylic, polyethylene, cotton, wool, any other suitable fiber composition, or a combination of such fiber compositions. Nonwoven material can have a weight in the range of about 0.25 ounces per square yard to about 1.0 ounces per square yard. In certain instances, the nonwoven material can have a weight in the range of about 0.3 ounces per square yard to about 0.5 ounces per square yard, about 0.25 ounces per square yard to about 0.6 ounces per square yard, about 0.4 ounces per square yard to about 0.7 ounces per square yard, about 0.6 ounces per square yard to about 1.0 ounces per square yard, or any other suitable range. An illustrative nonwoven material that can be used in the backing layer of the laminated article is a spunbonded polypropylene nonwoven material obtained from First Quality Nonwovens, Inc. (Great Neck, N.Y.).

Elastic fabrics can also be used. For example, various elastic warp knit fabrics are known, wherein non-elastic yarn is formed into a fabric or a mesh to bind and hold laid-in elastic threads within the structure in a stretched state to impart elastic properties to the fabric structure. Other elastic warp knit fabrics are known, wherein the structure is formed from stitches that have non-elastic and elastic thread components. Each individual elastic thread is a component of only one stitch in a course. Fabrics with laid-in elastic yarn can have a high incidence of streaks if the non-elastic yarn is knit with tension on the non-elastic yarn low enough to produce a soft hand-feel in the fabric. Fabrics with laid-in elastic yarn can be engineered to have good stretch and modulus properties in the length of the fabric, but generally they have lower stretch properties in the width of the fabrics. Fabrics with single strands of elastic yarn formed into stitches with the non-elastic yarn generally have a high incidence of streaks because of the non-consistent response of the elastic yarn in the stitches. They can also be more costly because they require larger quantities of expensive elastic yarn for a given fabric weight. They generally have relatively long stretch properties, but a relatively high modulus. Woven elastic fabrics are also known and may be used.

Warp knit elastic fabrics are also known, wherein a knitted ground construction composed of a plurality of pairs of non-elastic warp threads are formed into a plurality of wales and courses of single thread stitches, one thread of each of the pairs forming stitches in adjacent wales and alternate courses, and wherein the other thread of each of the pairs forms stitches in non-adjacent wales and alternate courses. A plurality of elastic threads extending between the wales generally parallel thereto are inlaid in the ground construction, with a non-elastic warp thread of the ground construction wrapped about each of the elastic threads to maintain the elastic threads in the ground construction. Other elastic warp knit fabrics are known, which include of a plurality of courses of elastic and non-elastic threads in which each of the elastic threads is knitted into every stitch across the width of the fabric in consecutive courses. Other elastic warp knit fabrics are known, wherein a ground construction that includes of a single non-elastic yarn system is used to bind and conceal laid-in elastic yarns from a single yarn system in such a way to reduce the danger of the non-elastic yarn in the knitted ground structure from raveling.

Elastic Layer

In articles that include an elastic layer, which can be part of the backing or separate from the backing, the elastic layer can include a sheet, yarn, and/or strand material that is capable of sustaining deformation without a permanent, detrimental loss of size or shape. Materials suitable for use as the elastic layer can be, e.g., elastic threads, yarn rubber, flat rubber (e.g., as bands), elastic tape, film-type rubber, polyurethane, tape-like elastomer, foam polyurethane or formed elastic scrim. The elastic layer can be unitary, multipart, or composite in construction. Threads or ribbons, where used, can be multiple and can be applied as a composite. The elastomerics used in the elastics can be latent and nonlatent.

Alternatively, stretch yarns, such as elastic stretch yarns or thermoplastic stretch yarns, can be used along the length of the fabric, preferably in the wale, to impart extensibility. Elastic stretch yarns, such as Lycra, Spandex, polyurethanes, and natural rubber, as described in, e.g., U.S. Pat. No. 4,668,563 (Buese), can also be used. Thermoplastic stretch yarns, such as polyesters and polyamides as described in, e.g., U.S. Pat. No. 4,940,047 (Richter et al.), can also be used.

The elastic strands described herein can be, e.g., a 210 denier spandex yarn, such as CREORA, (Hyosung, Inc., Korea and Hyosung (America) Inc., Rock Hill, S.C.). Another elastic yarn that can be used is, e.g., a 280 denier elastic yarn sold under the trademark RADICI SPANDEX (RadiciSpandex Corporation, Gastonia, N.C.). Depending on the amount of elasticity desired in the finished article, both the denier and number of elastic strands per inch (measured transversely) of the article may vary. For example, the denier of the elastic strands may vary from less than about 100 to about 1000, and the article can contain from about 5 to about 15 elastic strands per inch. In some articles, the elastic strands can be characterized by the ability to stretch from about 700% to about 800% of their original unstretched length before they fail. In some articles, the elastic strands can have a tensile strength of about 200 g to about 300 g in a standard tensile strength test. In some articles, the elastic strands can contribute about 4 g/m² to about 10 g/m² of the overall weight of the article, for example, about 6.5 g/m² of the article.

In articles that include an elastic layer, the elastic layer can be positioned between the backing layer and the foam layer, and can be permeated with a cohesive composition described herein. In other instances, the elastic layer can be secured to the foam and/or backing layers with other compositions or techniques. The elastic layer need not be used in concert with a backing layer, and can be secured to either side of the foam layer, as discussed above.

### Foam Wound Care Pads

In some instances, the foam layer cohesive article includes a foam pad that is distinct from a foam layer described above. The foam pad can be applied directly to an open wound, skin ulcer, or sore on a particular body part, and used to absorb fluids emitted from such wound, ulcer, or sore. The foam pad can be attached to a portion of the foam layer cohesive article, and in use the foam pad can be placed over the wound, sore, or ulcer, and the remainder of the article can be wrapped around the afflicted body part, for example twice or more. The article can compress the pad against the wound both securely and comfortably. The pad can also be used with other cohesive articles that do not necessarily include a foam layer, e.g., the CO-FLEX or POWERFLEX articles described above.

Foam wound care pads for direct application to an open wound include those described in, e.g., co-pending U.S. application Ser. Nos. 11/809,738; 11/809,766; and 11/809,469 (all of which are incorporated by reference). In one example, the foam wound care pad is attached to the foam layer elastic cohesive bandage, e.g., attached with an adhesive agent, and can be applied directly to an open wound or ulcer and secured in place by winding the foam layer cohesive bandage securely around the affected area.

Depending on the application, the foam pad can be hydrophilic and provide “wicking” of fluids, such as wound exudate, with which it is in contact. In particular, in many applications the foam pad can transport liquids such as wound exudate from the area of the wound itself through the foam, e.g., to the overlying surface of the foam layer cohesive bandage overwrap. The foam layer cohesive bandage overwrap can be adjacent to the surface of the pad that is opposite to the surface contacting the wound. The word “contact”, as used herein, can be used interchangeably with the term “fluid contact” to mean that the pad is capable of wicking fluids from the wound site regardless of the presence of an interface material, such as a stockinette or gauze, between the pad and the wound. Foam wound care pads are compatible with many such interface materials, as long as the material does not interfere with fluid contact between the wound site and the pad to the point where the foam pad would not serve its intended purpose.

Useful foam pads typically demonstrate significant, and preferably substantial, hydrophilicity, such as open cell polyurethane, polyethylene and silicone foam pads. The foam pads can be pliant, extensible, and/or have an open-celled structure. As used herein, the term “open-celled” refers to a foraminous structure having interconnecting or communicating orifices or cavities therein caused by a sufficient number of the wall membranes of the foam cells having been removed. Further, as used herein, the word “impregnated” and inflected forms thereof refer to the situation in which an agent is intermingled with and in surrounding relation to the wall membranes of the cells and the interconnected cells of the layer.

The foam pad can include any one of a number of extensible foams that are open-celled, such as polyether- or polyester-based polyurethane foams. In applications where the foam pad is intended to absorb exudate from a wound, the porosity of the foam pad is selected in order to absorb a sufficient amount of wound exudate. For example, the foam pad can have from about 10 to about 50 pores per centimeter (i.e., about 30 to about 120 pores per inch), or about 20 to about 40 pores per centimeter. As used herein, the term “pores per centimeter” refers to the average number of pores located along a linear centimeter of the foam sheet. The number of pores per linear centimeter can be determined in any number of ways known to those of ordinary skill in the art, e.g., by photomicrographic means, or by measuring the foam's resistance to air flow or a pressure differential, and using such information to calculate the approximate number of pores in the foam.

When the number of pores per centimeter is decreased below about 10, a foam may feel coarse or rough, and may not hold enough wound exudate or provide the necessary strength for the resulting pad or to retain the desired conformation. It will be understood, however, that the desired number of pores per centimeter parameter is related to the ability of the foam pad to absorb exudate so as to provide sufficient properties for use as a wound dressing pad. In some applications, the pad may not be intended to absorb exudate, in which case the number of pores per centimeter of the pad, or even whether the pad is hydrophilic, is not a significant consideration in selecting the pad for that application. Instead, the pad may be selected on the basis of its comfort against the skin or its thickness, for example.

The dimensions of the foam pad depend in large part on the intended use of the pad. For example, a foam layer cohesive article having a foam wound care pad can be prepared and packaged having dimensions intended for use in apposition to a particular type and/or size of body part. One dimension can relate to the thickness of the affected body part, i.e., the distance(s) between the major surface to be contacted with the body part, and the opposite surface thereto. The length of the overlying foam layer cohesive bandage can be adjusted accordingly. The dimensions of the foam pad required depend on the surface area of the wound or ulcer to be supported and/or treated, and can be varied as desired, as apparent to those of ordinary skill in the art. The foam wound care pad can generally be trimmed, as with a blade or scissors, or by grinding or abrading, or even by hand tear, to provide a desired size and shape. For example, an article intended to be applied to a finger can have a length of, e.g., 5-10″, suitable for multiple wraps around an average finger, and a pad of a length of, e.g., 1-2″, suitable for less than one wrap around an average finger. In other instances, e.g., an article intended to be wrapped around a torso might have a length of, e.g., 2-5 yards. These values are intended to be exemplary. In many instances, the article will have a length allowing at least two and possibly several wraps around a desired body part of average size (e.g., an average arm, leg, finger, or torso), and a foam pad that extends less than one wrap around the desired body part, although any desired sizes of the article or pad are possible. The article can have any desired width, e.g., 1″, 1.5″, 2″, 3″, 4″, and 6″ widths, although other sizes are possible.

In some articles, the foam pad can have a thickness of about 0.4 cm to about 5 cm, e.g., about 0.6 cm to about 2 cm, e.g., about 5/16″ (about 0.8 cm). In some instances, the foam sheet is not of uniform thickness, e.g., where a portion of a body part requires additional support or cushioning. The pad is, desirably, sufficiently dimensioned to encompass the area of the body part to be covered.

The foam pad can have a density in the range of about 0.02 g/cm³ to about 0.15 g/cm³, and most usefully, about 0.02 g/cm³ to about 0.07 g/cm³. Examples of suitable foam pads include “E-100”, “E-290”, “P-60”, “P-80” and “P-100”, each available from Illbruck U.S.A., Minneapolis, Minn. Another material that can be used for the foam pad is “E-150”, a polyether-based polyurethane foam sheet approximately 2 cm thick (available from Illbruck USA).

These hydrophilic foam compositions can be prepared by any means known in the art, such as by foaming prepolymers by means of the addition of chemical or physical blowing agents. Accordingly, hydrophilic polyurethane compositions can be prepared either by foaming isocyanate-capped prepolymers by the addition of water, or by frothing aqueous dispersions of fully reacted polyurethane polymers to entrap chemically inert gases therein. These foam compositions must be prepared, of course, with the understanding that any types or amounts of additives, introduced to confer or improve hydrophilicity or other characteristics of the foam, will not result in medically unacceptable cytotoxicity in the ultimate composition so produced. For example, the following surfactants can be used to enhance hydrophilicity in the preparation of hydrophilic foam compositions for use articles described herein: sorbitan trioleate; polyoxyethylene sorbitan oleate; polyoxyethylene sorbitan monolaureate, polyoxyethylene lauryl ether; polyoxyethylene stearyl ether; fluorochemical surfactants such as Zonyl FSN by E. I. du Pont and Fluorad FC 170C by 3M, and block copolymer condensates of ethylene oxide and propylene oxide with propylene glycol, such as the PLURONIC surfactants available from BASF Wyandotte.

In addition, the foam pad compositions can be thermoplastic, and thus reversibly soften upon heating. In some instances, the compositions will soften and become tacky, or at least self-adherent, between 225° F. and 300° F., although compositions can be used that soften between 200° F. and 350° F., and at the same time demonstrate thermal stability at ordinary room temperatures.

Further, the foam compositions can be cast or skived into low-density sheets. In particular, sheets formed from these compositions can have a density between 4 lb/ft³ and 20 lb/ft³, more usefully between 5 lb/ft³ and 12 lb/ft³, e.g., 8 lb/ft³. The low density of the foam pad contributes both to the lightweight absorbency of the foam bandage and the low cost of the materials necessary in the manufacture thereof. As discussed above, the low density foams can be open-celled or partially open-celled, as long as the foams are liquid permeable in contrast to the rigid impermeable closed-cell foams. However, the desired level of permeability will depend on the desired application.

Some useful foam pads include polyurethanes, including those that result from foaming isocyanate-capped prepolymers and those prepared by frothing aqueous polyurethane dispersions. Foam pads prepared by mechanically frothing, casting and curing aqueous polyurethane dispersions are also useful, e.g., foam pads recognized in the art as ionically water dispersible are particularly useful.

One useful system for preparing aqueous ionic polyurethane dispersions is to prepare polymers that have free acid groups, preferably carboxylic acid groups, covalently bonded to the polymer backbone. Neutralization of these carboxyl groups with an amine, preferably a water soluble monoamine, affords water dilutability. Careful selection of the compound bearing the carboxylic group must be made because isocyanates, the reactive group employed most often in the generation of urethane linkages, are generally reactive with carboxylic groups. However, as disclosed in U.S. Pat. No. 3,412,054 (which is incorporated herein by reference), 2,2-hydroxymethyl-substituted carboxylic acids can be reacted with organic polyisocyanates without significant reaction between the acid and isocyanate groups as a result of the steric hindrance of the carboxyl by the adjacent alkyl groups. This approach provides the desired carboxyl-containing polymer with the carboxylic groups being neutralized with the tertiary mono-amine to provide an internal quaternary ammonium salt and, hence, water dilutability.

Suitable carboxylic acids and, preferably, the sterically hindered carboxylic acids, are well-known and readily available. For example, they can be prepared from an aldehyde that contains at least two alpha position hydrogens that are reacted in the presence of a base with two equivalents of formaldehyde to form a 2,2-hydroxymethyl aldehyde. The aldehyde is then oxidized to the acid by procedures known to those of ordinary skill in the art.

The polymers with the pendant carboxyl groups are characterized as anionic polyurethane polymers. However, an alternate route to confer water dilutability is to use a cationic polyurethane having pendant amino group. Such cationic polyurethanes are disclosed in, e.g., U.S. Pat. No. 4,066,591, which is incorporated herein by reference.

Useful polyurethanes can be made, e.g., by reacting di- or polyisocyanates and compounds with multiple reactive hydrogens suitable for the preparation of polyurethanes. Such diisocyanates and reactive hydrogen compounds are disclosed in U.S. Pat. Nos. 3,412,054 and 4,046,729, the entire contents of which are incorporated herein by reference. Further, the processes to prepare such polyurethanes are well known in the art. Aromatic, aliphatic and cyclo-aliphatic diisocyanates or mixtures thereof can be used in forming the polymer. Such diisocyanates, for example, for tolylene-2,4-diisocyanate; tolylene-2,6-diisocyanate; meta-phenylene diisocyanate; biphenylene-4,4′-diisocyanate; methylene-bis-(4-phenol isocyanate); 4,4-chloro-1,3-phenylene diisocyanate; naphthylene-1,5-diisocyanate; tetramethylene-1,4-diisocyanate; hexamethylene-1,6-diisocyanate; decamethylene-1,10-diisocyanate; cyclohexylene-1,4-diisocyanate; isophorone diisocyanate and the like. Arylene and cycloaliphatic diisocyanates are particularly useful.

In some instances, the polyurethane foam can be produced using a dispersion viscosity that is generally in the range of from 10 centipoise to 1000 centipoise. Useful solutions of polyurethane in organic solvents, by contrast, generally have viscosities of several thousand centipoise, ranging as high as 50,000 centipoise when the solution contains about 20% to about 30% by weight polyurethane. Useful polyurethane dispersions contain, moreover, about 50% to about 75% percent by weight polyurethane solids in dispersion. A particularly useful polyurethane concentration is 55% to 70% by weight and the most preferred concentration is 65% by weight polyurethane solids in dispersion.

Particularly useful polyurethane dispersions include the non-crosslinked polyurethane compositions recited in U.S. Pat. No. 4,171,391, incorporated herein by reference. Other useful polyurethane dispersions include those available from Witco Chemical Company under the trade designation Witcobond® W-290H; these dispersions yield foams that demonstrate inherent hydrophilicity, even in the absence of surfactants. The Witcobond® W-290H dispersions contain 65% by weight anionic polyurethane solids having particulate diameters less than 5 μm.

Use of Cohesive Article with Unna Boot Medicated Pad

Foam layer cohesive articles can also be used to secure a medicated “Unna boot” pad to a body part. An Unna boot is a moist, gauze bandage carrying calamine lotion and, optionally, zinc oxide and/or glycerine. The original Unna boot was first described in 1854 and named for its inventor. The Unna boot medicated pad promotes healing of ulcers, such as venous ulcers, by reducing infection and increasing the return of blood to the heart. For venous leg ulcers, the Unna boot is wrapped from the toes to just below the knee, covering the ulcer and the lower leg. The gauze then dries and hardens.

Conventional latex-free cohesive articles are generally incompatible with Unna boots, as is well known in the art. If a conventional latex-free cohesive article is attempted to be used to secure an Unna boot to a body part, the article rapidly loses its cohesive properties and subsequently unravels. Specifically, as the calamine lotion seeps through the article, it breaks cohesive bonds between overlying layers of the wrapped article. In contrast, a foam layer cohesive article—even one with a latex-free cohesive composition—can be successfully used with an Unna boot. The foam layer cohesive article satisfactorily retains its cohesion when used to wrap an Unna boot to a body part. Likewise, the cohesive compositions of the present invention may be successfully used with an Unna boot as described herein.

Without wishing to be bound by theory, it is believed that one factor contributing to the successful use of the foam layer cohesive article with an Unna boot is that the foam layer in the article slows the speed of the lotion in the Unna boot from seeping through the article as it is being wrapped around the body part. This may allow the cohesive composition in the article to form a cohesive-to-cohesive contact between underlying/overlying layers of the article, and then the tiny “suction cups” in the open cell structure of the foam form a secure cohesive bond to underlying/overlying layers, and the lotion cannot penetrate these bonds.

Thus, the foam layer cohesive article may be wrapped snugly over the Unna boot. A foam layer cohesive bandage/Unna boot dressing can be applied, e.g., every one to two weeks, until the ulcer is healed. Initially, more frequent changes may be required for heavily draining ulcers.

An exemplary Unna boot pad is the GELOCAST™ Unna's Boot Dressing, which is a non-raveling gauze preparation carrying a soothing zinc oxide/calamine formulation that provides firm compression therapy promoting the healing of irritated or ulcerated skin (BSN-Jobst Gelocast Unna Boot Dressing −4″×10 yards, available from the Medical Supply Company (Alpharetta, Ga.)).

Other Unna boot preparations include the Unna's Boot commercially available from Biersdorf, Inc., which includes a zinc paste-containing bandage wrapped around a patient's leg from above the toes to below the knee. Still other Unna's Boot/zinc impregnated treatments are available from Miles and Graham Field. These dressings are often left in place for a week at a time and typically require the use of absorbent pads that must be applied to the outside of the dressings in the area of the ulcer to absorb excess exudate. Seepage of exudate throughout the wrap is common, and damage to the skin and epithelium may occur. The foam layer cohesive bandages described herein are capable of absorbing this fluid, thereby providing therapeutic pressure to the wound while obviating the use of additional absorbent dressings.

### Optional Sterilization of Foam Layer Cohesive Articles

Foam layer cohesive articles can optionally be sterilized using ethylene oxide (EtO) techniques known in the art, without detrimentally affecting the properties of the article. Typically, the well-known EtO process includes four basic phases: (1) air removal (vacuum), (2) steam injection and conditioning dwell, (3) EtO injection and gas dwell, and (4) gas purge and air inbleed. In the case of a conventional, rolled cohesive bandage, the multiple pressure and vacuum operations involved in the EtO process typically cause the bandage roll to shrink in size and compress, and can greatly increase the cohesive bond between overlying and underlying layers. If the bandage peel values become too high as a result of the sterilization process, the bandage will be extremely difficult if not impossible to remove from the roll. Thus, in order to limit the effects of this “squeezing” during the vacuum portion of the EtO process, conventional cohesive bandages are generally manufactured using lower peel values and rolled looser than manufacturers normally would for a bandage that would not be subjected to the EtO process.

In contrast, foam layer cohesive articles do not have this limitation. Without wishing to be limited by theory, it is believed that the closed cells that are inherently present in a foam (even an open-cell foam, as described above and as is known in the art) expand during the vacuum portion of the EtO process, and that this expansion keeps the individual layers of the foam separated when in roll form. This expansion may also keep the open cells separated, which may otherwise have caused compression of the bandage. This feature, among other possible features, allows the bandage made with a foam layer to be manufactured using normal peel values and wound to normal tension levels.

After sterilization, the article can be packaged so as to maintain its sterility until use, using techniques that are known in the art. The article can also be packaged without requiring a sterilization step, e.g., using a flow wrap for a non-sterile product, or a Dupont Tyvek® 1059B for a sterile product.

### Pressure-Sensitive Articles

Pressure-sensitive articles can include adhesives that adhere to most surfaces with very slight pressure and they retain their tackiness. Such pressure-sensitive articles include those described in, e.g., US Publ. No. 20050158539 and 20070259163.

Pressure-sensitive adhesives include a large group of adhesives that utilize many different polymers (acrylics, rubbers, polyurethanes, silicones or siloxanes), together with plasticizers and tackifying resins to form a permanently tacky (sticky) adhesive. The name “pressure-sensitive” comes from the fact that moderate pressure alone is sufficient to spread the viscous adhesive layer on to the surface to be adhered to and achieve useful adhesive strength. They are available in solvent, hot-melt, latex, and water based forms. Pressure sensitive adhesives are often based on non-crosslinked rubber adhesives, acrylics or polyurethanes. They form viscoelastic bonds that are aggressively and permanently tacky, and adhere without the need of more than finger or hand pressure.

Generally, suitable pressure sensitive adhesives include, for example, those based on natural rubbers, synthetic rubbers, styrene block copolymers, polyvinyl ethers, poly (meth)acrylates (including both acrylates and methacrylates), polyurethanes, polyureas, polyolefins, and silicones. The pressure sensitive adhesive may comprise an inherently tacky material, or if desired, tackifiers may be added to a tacky or non-tacky base material to form the pressure sensitive adhesive. Useful tackifiers include, for example, rosin ester resins, aromatic hydrocarbon resins, aliphatic hydrocarbon resins, and terpene resins. Other materials can be added for special purposes, including, for example, plasticizers, hydrogenated butyl rubber, glass beads, conductive particles, filler, dyes, pigments, and combinations thereof.

Any pressure-sensitive adhesive is useful for preparing the articles of the invention. Pressure-sensitive adhesives generally include elastomers that are inherently tacky or elastomers or thermoplastic elastomers that include tackifying resins and plasticizing additives. Fillers, antioxidants, stabilizers and crosslinking agents known in the art also may be used. A fluid, typically water, is added to reduce the viscosity to a level that is easily applied to the open fabric. The amounts and kinds of ingredients of the pressure-sensitive adhesive are selected to provide appropriate substrate adhesion and target peel strength. Strong substrate adhesion and a moderate peel strength are desired for use with living skin. Suitable pressure-sensitive adhesives include polyacrylate adhesives, polyalphaolefin adhesives, such as linear, radial, branched and tapered block copolymers including styrene-butadiene, styrene-ethylene/butylenes and styrene-isoprene block copolymers, polyvinyl acrylates, natural and synthetic rubber resin adhesives, silicones, polydiorganosiloxane polyurea copolymers, and mixture and blends thereof. Many suitable pressure-sensitive adhesives are known in the art and may be utilized with the methods and compositions described herein. Particularly useful pressure-sensitive adhesives include acrylic resins (e.g., Gelva™ Multipolymer Solution 2495; Cytec Surface Specialties; Indian Orchard, Mass.).

The adhesive can be located on upper and/or lower surfaces of the article (e.g., an open fabric or a film). Where the article is or includes a fabric, the pressure-sensitive adhesive may cover optionally the upper and lower surfaces without spanning adjacent yarns, so that porosity or openness is retained. Where the article is or includes a film, the pressure-sensitive adhesive may cover either of both surfaces of the film. The adhesive may also be suffused or permeated throughout the entire thickness of the open fabric of an article. The pressure-sensitive adhesive may be selected to be removable from the skin without separation of the substrate backing from the open fabric.

A pressure-sensitive adhesive article can include a porous backing having an adhesive-carrying open fabric adhered thereto. The open fabric can be of an open weave or knit and the adhesive can be located only on the fabric yarns, threads or fibers without spanning or bridging of the adhesive between yarns, threads or fibers. In this way, the porosity of the backing is maintained so that a breathable article having high vapor permeability is obtained. In some embodiments, the adhesive penetrates a distance into the backing substrate to anchor the open fabric to the backing. In some other embodiments, the open layer is of unequal tensile strength in the cross and machine directions and thereby imparts different tear characteristics to the article in the machine direction (MD) and cross direction (CD). The open fabric provides sufficient strength to the article in the machine direction so that the tape does not fail during use; however, the strength of the tape in the cross direction permits an even and easy tear. In one or more embodiments, the tape is hand tearable. In still other embodiments, the pressure-sensitive adhesive article exhibits two or more of these features.

By “open structure” it is meant that the weave includes areas that are open or free of yarn or fibers (and adhesive). The open structure can include pores such as are typically found in non-woven fabrics, or it can be a much larger open structure such as a scrim or mesh. The openness of a structure is defined, for example, by pore size, thread count and/or % open area.

The backing substrate is any conventional porous backing and can be a woven fabric, knit fabric, non-woven fabric, or film. The backing fabric is not required to be of high tensile strength because the open fabric provides tensile strength in both the cross and machine directions. The porosity of the backing substrate is sufficient to provide a breathable, water vapor permeable membrane in the assembled pressure-sensitive tape. The backing substrate can be more than about 25% open area, and more than about 50% open area in some embodiments.

In a non-woven substrate backing, the fibers are intimately entangled with each other to form a coherent, breathable fibrous non-woven backing. The particular fiber composition used as a non-woven backing substrate is selected from those known in the prior art, according to the web property desired. For example, the non-woven substrate backing may be selected from the naturally occurring animal and vegetable fibers, including cotton and wool, or synthetic (chemical) fibers such as nylons, cellulosics, rayon, polyesters, polyamides, acrylics, polypropylene, polyethylene, and the like, including blends of such fibers. In one or more embodiments, the nonwoven fabric is lightweight and can typically be about 10-20 grams per square meter.

The non-woven substrate backing can further include a bonding agent or sizer to lock adjacent fibers of the non-woven fabric. The bonding agent promotes adhesion of the pressure-sensitive adhesive to individual yarns or fibers of the substrate backing when the pressure-sensitive adhesive and the backing are combined. Suitable bonding agents are selected from those known in the art, and can include, by way of example, homopolymers and copolymers of synthetic latexes such as butadiene, acrylics, vinyls and the like. The bonding agent is applied from a liquid carrier or solution at low solids levels so that the porosity of the non-woven is not impaired. The manner of applying the binding agent to the non-woven web is non-critical and any of the known methods of the coating art may be employed. Commercially available bonded non-woven fabrics can also be used in the articles of the present invention.

Woven or knit fabrics can also be used as a backing substrate and are selected from those known in the prior art. Exemplary fabrics include woven cotton fabrics, woven rayon, polyester or polypropylene fabrics and knit fabrics such as polyester, polypropylene and nylon knit fabrics.

The porous fabric having an open structure can be a woven or knit fabric. The openness of the fabric (which is a function of, for example, thread count and yarn denier) is selected so that the assembled structure, e.g., backing substrate, adhesive, and open fabric, is porous and vapor permeable. It is also selected to provide sufficient adhesive surface area to establish a strong adhesive contact with the backing substrate. The fabric can be up to about 95% open, i.e., 5% of surface area of the article is porous fabric, and is typically at least about 50% open. By way of example only, the open fabric can be an open weave fabric such as gauze, e.g., cotton or synthetic polymer gauze, or a warp-knit fabric.

In some embodiments, the open fabric exhibits a tensile strength differential in the machine and cross directions of the fabric. In order to provide warp and weft yarns of different tensile strength, yarns of different denier can be used. Denier is a unit of fineness for yarns, based upon 50 milligrams per 450 meters of yarn (1 denier). For fabrics using warp and weft yarns of the same or different material, differences in tensile strength can be achieved by using yarns of different denier, e.g., a “thin” yarn and a “thick” yarn. By way of example only, warp yarns of about 40-60 denier and weft yarns of about 70-150 denier have been used. In other embodiments, different warp and weft strengths are achieved by using yarns of different filament counts. By way of example only, a low denier monofilament is used as a warp yarn and a high denier multifilament yarn is used as the weft yarn.

In one or more embodiments, a knitted fabric can be used, in which the yarns are formed into stitches in a lengthwise (machine) direction and a weft (cross machine) insert yarn of same or different strength is inserted through the warp stitches to provide a fabric having the same or differing tensile strengths in the warp and weft directions. In some embodiments, the warp knit/weft insertion fabric has a weight of less than about 50 grams per square meter (about 1.5 oz. per square yard) or about 25-30 grams per square meter (about 0.7-0.9 oz. per square yard), and may be as low as 5 grams per square meter. An exemplary warp knit/weft insertion fabric has a weight ranging from about 25 to about 10 grams per square meter, and a warp/weft thread count ranging from about 18×12 to about 9×12. The knitted warp yarns are about 40 denier polyester, and the about 150 denier fill or weft yarns are loose, non-twisted, textured polyester filaments. Similar warp knit/weft insertion fabrics are available commercially, e.g., warp knit/weft insertion greige fabric is available from Milliken & Company of Spartenburg, S.C. A warp knit/weft insertion construction provides a lightweight fabric having high tensile strength, e.g., about 12-13 lb/in², in the warp direction.

In one or more embodiments, the open fabric is characterized by a warp yarn(s) of lower tensile strength than the weft yarn(s). The difference in tensile strength gives rise to different tear characteristics in the cross or machine directions; and the arrangement of the weave provides a clean, even tear along the CD. The low stretch characteristics of the MD yarns tend to focus the load at the point of tear and cause the yarns to fail in a predictable manner. The stronger CD yarns tend to guide the tear and cause the tear to propagate between the CD yarns. The CD yarns also promote a straight tear across the structure and cause the fibers (of the nonwoven backing substrate) to break cleanly without a ragged, uneven edge.

In some embodiments, the pressure-sensitive adhesive tape can include elastic yarns, resulting in a self-wound pressure-sensitive tape having a degree of stretch (elongation) ranging from approximately 30% to 150%. The backing substrate and the open fabric can have substantially the same elasticity and extensibility.

The adhesive-coated open fabric can adhere to the backing substrate by adhesive contact. Adhesion of the open fabric to the substrate can be enhanced by partial penetration of the adhesive into a portion of the thickness of the backing substrate. Adhesive can be absorbed by the backing only in those areas where the open fabric contacts the substrate. The open areas of the open fabric can be substantially free of adhesive, so that no adhesive is transferred to the backing substrate in these areas. The adhesive does not saturate the full thickness of the backing, so that the side of the backing substrate opposite the open fabric is essentially free of adhesive. The two different tape surfaces make the pressure-sensitive tape self-winding and permit an even unwind of the tape from a roll. The adhesive can penetrate up to about 95% of the thickness of the backing substrate, and in some embodiments, the adhesive penetrates into about 25% to about 75% of the backing thickness. Typically, the adhesive penetrates about 50% of the backing thickness.

Because the adhesive-carrying open fabric retains its openness, the vapor permeability of the article remains high. Microporosity and water vapor permeability can be measured in a variety of ways, for example, by measuring the amount of air expressed in mL/min by a known surface at a certain pressure. Pressure-sensitive adhesive tapes desirably maintain a maximum water vapor transmission rate. An exemplary tape prepared according to one or more embodiments of the invention had a water vapor transmission (WVT) of 28 grains/ft²-h (water method) (ASTM: E96-00^{ε}_{¹}), which represents at least about a 25% improvement over current industry standards.

In some embodiments, the pressure-sensitive articles include backings with a releasable outer surface as described in, e.g., US Publ. No. 20070259163. One surface of an adhesive article, according to certain embodiments, includes a fabric carrying a pressure-sensitive adhesive, to form an adhesive layer. The other surface of the article includes a non-woven fabric carrying a binder, to form a backing layer that is applied to and coextensive with the adhesive layer. The backing layer is bonded or laminated to the adhesive layer, so that the two layers do not come apart during use, e.g., while the article is being used as supportive ankle strapping. However, the backing has particularly useful adhesive properties so that, when the article is wound up to form a roll, application of a light force, e.g., hand force, is sufficient to unwind the roll. Accordingly, the backing might be said to have a “non-stick” characteristic, although this is to be understood to mean that the backing allows firm, albeit easily reversible, adhesion to the pressure-sensitive adhesive layer side of the article such that the roll does not spontaneously unwind, but is easily unwound by a human user. Furthermore, while overlying layers of the article in the roll separate easily from one another, the binder and adhesive layer of any given layer of the article do not separate from each other during unwind. Additionally, the roll does not inadvertently unwind without the use of sufficient force, making it easy to transport and handle. In summary, the adhesive article can be wound into a roll, easily handled as a roll, and subsequently unwound and used while maintaining structural integrity. Moreover, the adhesive article is soft to the touch and comfortable to use against skin. The article is also typically breathable and provides a high water vapor transmission rate to prevent sweat-induced failure and/or skin maceration, particularly for athletic applications. The article is typically also pliable and/or conformable.

The types and amounts of materials used in the backing and the adhesive layer impart various characteristics to the finished article. The fabrics used in the backing and the adhesive layer allow the article to be conveniently torn by hand in a direction transverse to and/or longitudinal to the direction of the article. An article intended for use as a supportive strapping tape may be made substantially non-elastic by selecting a backing material that is relatively non-elastic, or even by incorporating non-elastic materials into the article, such as a warp-knit weft-insertion or woven scrim fabric. Or, an article intended for use as a stretch bandage can be made elastic by including an elastic layer in the article. These and other adhesive articles with “non-stick” backings, and methods of making same, are described in greater detail below. Additionally, some standard methods of testing the mechanical characteristics of adhesive articles, and the results of these measurements on finished adhesive articles with “non-stick” backings, fabricated according to certain embodiments, are also described.

### Adhesive Formulations

Exemplary adhesive formulations that can be used in the methods and compositions described herein are also known in the art and described in, for example, U.S. Pat. No. 4,112,213, U.S. Pat. No. 4,917,928, U.S. Pat. No. 4,917,929, U.S. Pat. No. 5,141,790, U.S. Pat. No. 5,045,386, U.S. Pat. No. 5,229,207, U.S. Pat. No. 5,296,277, U.S. Pat. No. 5,670,557, U.S. Pat. No. 6,232,366, and U.S. Publication No. 2005/0249791, the disclosures of which as incorporated herein by reference in their entireties.

The invention is further illustrated by the following examples. The examples are provided for illustrative purposes only. They are not to be construed as limiting the scope or content of the invention in any way.

### EXAMPLES

### Example 1

### Production of an Antimicrobial Cohesive Article

### Method I

20% solids aqueous solution of IonPure WPA (≦10 microns) soluble glass beads (Ishizuka Glass Co., Ltd. of Japan), ACT T 558, or ACT Z 200® (EnviroCare Inc., Wilmington, Mass.) and either Dispercoll C74 (available from Bayer Material Sciences, LLC) or Airflex 405 (available from Wacker Polymers) were mixed. To this mixture, 1.0% of Zetasperse 2300 (available from Air Products) was added as a dispersant or wetting agent to aid in the homogeneous dispersion of the IonPure soluble glass beads or the ACT T 558® or ACT Z 200® zeolites. For knife over roll applications, 4% of the active was admixed portion-wise followed by the addition of Rheolate 1 or Rheolate 360 (available from Elementis). The Dispercoll solution and the Airflex solution were then sprayed, dipped and nipped, gravure coated or printed onto CoFlex NL bandages (Andover, Salisbury, Mass.).

### Method II

In another method, 50% solids aqueous solution of IonPure WPA (≦10 microns) soluble glass beads (Ishizuka Glass Co., Ltd. of Japan) or ACT T 558® or ACT Z 200® (EnviroCare Inc., Wilmington, Mass.) zeolites and either Dispercoll C74 or Airflex 405 were mixed. To this mixture, 1.0% of Zetasperse 2300 was added as a dispersant or wetting agent to aid in the homogeneous dispersion of the active material. To this, 1% Unifroth 1672 (available from Unichem, Inc.) was added as a foaming surfactant. Using the Chemical Foam System (CFS) method (see EP 0995826 B1), the Dispercoll solution and the Airflex solution were then added to CoFlex NL bandages (Andover, Salisbury, Mass.). Then 4% of the active was admixed portion-wise, after which compressed air was added to initiate the formation of ultra thin walled bubbles which burst and then coalesced into a contiguous thin film. The target coating weight of the active containing film forming resin was 0 gsm>x<10 gsm.

### Example 2

### Cohesive Properties of an Antimicrobial Cohesive Article

### Method

The cohesiveness of Andover's CoFlex NL with Silver I (prepared as described in Example 1, Method II) was tested using a 180 degree peel bond (front-back) procedure. Equipment used were a Thwing-Albert Material Tester QC-1000 (West Berlin, N.J.); a die cutter with 1″×4″ die (Masterlog sample) or 1″×12″; template (pre-cut rolls); self-healing mat marked in inches; Cheminstruments roll-down machine with 10 lb rollers (for bandage evaluation) or 4.5 lb rollers (for tape evaluation); and release paper.

For some tests, the article was cut using the die cutter into strips 1″×4″ long. For other tests, approximately two feet were removed from the beginning of a roll and discarded, and cut into strips 1″×4″ long using the marked healing mat. One piece was laid on top of the other, and care was taken to ensure that the pieces were joined front to back. Release paper was then inserted between the layers at one end for separation after rolling. For bandages, the 4″ sample was then rolled with the 10-pound roller four times back and forth at a rate of 12 inches per minute each direction. For tape, the 4″ sample was rolled with the 4.5-pound roller five times back and forth at a rate of 12 inches per minute each direction. The peel properties of the samples were then tested using the Thwing-Albert Material Tester QC-1000 according to the manufacturer's protocol.

### Results

The results of the peel tests are listed in Table 1. The specification range for CoFlex NL with Silver I (prepared as described in Example 1, Method II) is 12 oz/in to 20 oz/in. Little reduction in the cohesiveness of the product was observed with the addition of the IonPure Soluble Glass Beads or ACT zeolites at 10% & 20% based upon the solids of the film forming resin. There was also little difference in the effect of the film forming resin on this property whether it was chemically similar (i.e., IonPure or ACT zeolites in a layer of Dispercoll Polychloroprene) or dissimilar (i.e., IonPure or ACT Zeolites in a layer of AirFlex VAE).

TABLE 1
Results of Peel Test

| | | Tan Non Latex + | Tan Non Latex + | Tan Non Latex + |
|---|---|---|---|---|
| | | (Surface Coating) | (Surface Coating) | (Surface Coating) |
| | Tan Non | 10% SilverI/20% Dispercoll | 20% SilverI/20% Dispercoll | 10% SilverI/ |
| | Latex Control | Polychloroprene | Polychloroprene | 20% AirFlex VAE |
| Peels | 17.4-18.0 | 16.0-18.3 | 14.0-14.9 | 14.5-15.5 |
| Front/Back oz/in | | | | |
| Stretch Coated | 75-80 | 83-88 | 84-85 | 82-86 |
| Weight (gsm) | | | | |
| % Stretch | 135-137 | 145-149 | 133-135 | 141-149 |

### Example 3

### Antimicrobial Properties of an Antimicrobial Cohesive Article

### Method

CoFlex LF2 (Andover Healthcare, Inc., Salisbury, Mass.) with Silver I was prepared as described in Example 1, Method II. A sample was cut into a 2″×4″ piece of material, which was folded back onto itself and the silver stripes on the exterior surface (which were now on the interior surface) were firmly pressed together. This resulted in a 2″×2″ piece, which was placed into a flask. 57 mL of a culture of Methicillin Resistant Staphylococcus aureus (MRSa) was diluted in Butterfield's Buffer and added to the flask. The numbers of survivors of MRSa at time=0 and after 24 hours exposure were determined in triplicate by measuring the number of colony forming units (CFU).

### Results

At time=0, replicate 1 yielded 5.2×10⁵ CFU/mL (5.72 log₁₀); replicate 2 yielded 5.1×10⁵ CFU/mL (5.72 log₁₀); and replicate 3 yielded 2.48×10⁵ CFU/mL (5.394 log₁₀). The average number of survivors was 4.07×10⁵ CFU/mL (5.61 log₁₀).

24 hours after exposure, replicate 1 yielded 2.9×10² CFU/mL (2.46 log₁₀); replicate 2 yielded 7.1×10² CFU/mL (2.85 log₁₀); and replicate 3 yielded 1.10×10³ CFU/mL (3.041 log₁₀). The average number of survivors was 6.03×10² CFU/mL (2.78 log₁₀).

Control flasks yielded 4.8×10⁵ CFU/mL (5.68 log₁₀) at time=0, and >2.000×10⁷ CFU/mL (>7.3010 log₁₀) at 24 hours.

It should be noted that one would expect significant growth in both the test and control flasks because the test organism mixture added to each flask contained approximately 10% growth media and 10% fetal bovine serum. Furthermore, each flask (Test and Control) was held at 35-37° C. for the desired exposure time. It was demonstrated that over the course of the test the control flask grew from 4.8×10⁵ CFU/mL (5.68 log₁₀) at time=0 to >2.000×10⁷ CFU/mL (>7.3010 log₁₀) at 24 hours. CoFlex LF2 with Silver I demonstrated a >99.99% (>4.52 log₁₀) reduction of MRSa survivors as compared to the number of survivors in the control flask following a 24 hour exposure period when tested at 35-37° C. in the presence of a 10% fetal bovine serum organic soil load. Using a control flask for comparison provides the most accurate assessment of the test articles in the real world, and these data clearly reflect both the bactericidal and inhibitory activity of Silverl against MRSa.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antimicrobial article comprising a substrate and a silver compound, wherein the silver compound is present in an amount sufficient to treat the growth or spread of an infectious agent through cutaneous contact of the antimicrobial article with a subject.

2. The article of claim 1, wherein the silver compound is a glass bead or a natural zeolite containing silver.

3. The article of claim 1, wherein the article comprises a first surface comprising the silver compound.

4. The article of claim 3, wherein the substrate comprises a formulation comprising the silver compound, and wherein the formulation is applied to the first surface of the substrate.

5. The article of claim 4, wherein the formulation comprises a cohesive agent.

6. The article of claim 4, wherein the formulation comprises an adhesive agent.

7. The article of claim 4, wherein the formulation comprises a pressure-sensitive adhesive agent.

8. The article of claim 1, wherein the article is a tape.

9. The article of claim 1, wherein the article is a bandage.
